(19) United States
(12) Patent Application Publication
(10) Pub. No.: US 2026/0191552 A1
(43) Pub. Date: Jul. 9, 2026

(54) DYNAMIC MEDICAL TOOL FOR TISSUE PENETRATION

(71) Applicant: BIONAUT LABS INC., Los Angeles, CA (US)

(72) Inventors: Florent CROS, Los Angeles, CA (US); Michael SHPIGELMACHER, Los Angeles, CA (US)

(73) Assignee: BIONAUT LABS INC., Los Angeles, CA (US)

(21) Appl. No.: 19/440,174

(22) Filed: Jan. 5, 2026

Related U.S. Application Data
(60) Provisional application No. 63/742,333, filed on Jan. 6, 2025.

(51) Int. Cl. A61B 17/32; A61B 17/00; A61B 34/30

(52) U.S. Cl. CPC A61B 17/320068; A61B 34/30; A61B 2017/00017; A61B 2017/00853; A61B 2017/00876; A61B 2017/320082

(57) ABSTRACT

Provided are medical tools for improved surgical function. The medical tools are coupled with a means of dynamic motion such as vibration for use on a patient. Systems and methods related to the tool are also disclosed herein.

### CROSS REFERENCE TO RELATED APPLICATIONS

This application claims the benefit of priority of U.S. Provisional Application No. 63/742,333, filed Jan. 6, 2025, hereby incorporated by reference.

### FIELD OF THE INVENTION

The presently disclosed subject matter relates to the field of medical tools for improved surgical function. The medical tool is coupled with a means of dynamic motion such as vibration for use on a patient. Systems and methods related to the tool are also disclosed herein.

### BACKGROUND

In a variety of applications there is a need for precise careful penetration of medical tools into tissue. Examples include penetration of needles for injection, insertion of brain shunts into brain tissue, insertion of electrostimulation tools, etc. There is a correlated relationship between the force exerted on the tool and the damage to tissue. In addition, increased insertion force has been correlated with pain. Finally, exertion of strong forces for insertion may increase procedure risk as the tool moves from a high force medium to a low force medium, increasing the risk of accidental motion overshoot. For these reason, there is medical benefit to control insertion force with greater resolution, and allow penetration of biological tissue at lower force levels in general.

Multiple dimensions can be optimized to lower the penetration force for a medical tool. These include:
Tool size and geometry (e.g., angle of needle tip);
Specific surface coatings to minimize friction with tissue (e.g., hydrophilic coatings);
Surface modifications (e.g., micro etching);
Insertion speed (e.g., certain biological tissue exhibit greater resistance for higher insertion speed);
Axial vibration of needles in the direction of motion.

However, no work has been done to analyze vibration of the inserted tool in the diametrical axis (orthogonal to the axis of insertion).

This present disclosure describes a system and a method to allow diametrical vibration of the inserted tool in an axis orthogonal to the direction of insertion.

### SUMMARY

In one embodiment the invention provides a dynamic medical tool for use in an individual, the dynamic medical tool comprising:
a medical tool comprising a distal end;
an element comprised in the medical tool;
wherein the element comprises a magnetic component, an actuator, or a combination thereof; and
wherein the element is configured to induce motion of the medical tool.

In one embodiment the medical tool is selected from: catheter, magnetic capsule robot, endoscope, hypodermic needle, hollowed needle, solid needle, trocar, cannula, scalpel, catheter, implant, stent, ultrasound probe, temperature probe, medical tube, cauterizing probe, and medical drill. In one embodiment the motion is selected from: vibration, linear motion, rotation, back-and-forth, circular, helical, pulsating, axial, lateral, and longitudinal. In one embodiment the element is located on the distal end of the medical tool, or in close proximity to the distal end of the medical tool. In one embodiment the range of motion of the distal end ranges between 0.1 mm to 5 cm. In one embodiment the magnetic component is selected from: permanent magnet, non-permanent magnet, and electromagnet, or a combination thereof. In one embodiment the permanent magnet comprises any of the following selected from: neodymium, samarium-cobalt, alnico, iron, steel, cobalt, and ceramic magnet. In one embodiment the device further comprises a friction-minimizing coating. In one embodiment the medical tool has a width ranging between 0.1 mm and 2 cm. In one embodiment the length of the medical tool ranges between 100 microns and 2 m. In one embodiment the induced motion reduces friction, enhances ease of penetration, or a combination thereof, when the dynamic medical tool is in contact with the individual. In one embodiment the dynamic medical tool is disposable.

In one embodiment the invention provides a dynamic medical tool system comprising:
the dynamic medical tool of the invention;
a controller configured to communicate with the dynamic medical tool; and
a power source.

In one embodiment the power source and controller are configured to operate the actuator to achieve the induced motion. In one embodiment the system further comprises a magnetic inducing apparatus configured to be operated to generate a varying magnetic field, thereby remotely controlling the motion of the dynamic medical tool. In one embodiment the magnetic field operates at a frequency ranging between 1 Hz to 5 kHz. In one embodiment the actuator operates at a frequency ranging between 1Hz to 5 kHz. In one embodiment the power source is located externally to the dynamic medical tool, comprised within the dynamic medical tool, or a combination thereof.

In one embodiment the invention provides a dynamic medical tool system for use in an individual, the dynamic medical tool comprising:
a magnetic capsule robot;
a magnetic inducing apparatus configured to be operated to generate a varying magnetic field, thereby remotely causing the magnetic capsule robot to vibrate;
a controller; and
a power source.

In one embodiment the vibration reduces friction, enhances ease of penetration, or a combination thereof, when the magnetic capsule robot is in contact with the individual. In one embodiment the magnetic field operates at a frequency ranging between 1 Hz to 5 kHz. In one embodiment the magnetic capsule robot further comprises a wireless communication module configured to transfer data between the magnetic capsule robot and the controller.

In one embodiment the invention provides a dynamic catheter comprising:
a catheter tube;
at least one embedded control wire extending longitudinally along the central axis of the catheter tube wire and configured to deflect the distal tip of the catheter tube upon application of tension to the at least one embedded control wire;
a vibrating element coupled to the at least one embedded control wire, the catheter tube, or a combination thereof, and configured to induce vibrations at the distal end of catheter tube.

In one embodiment the dynamic catheter further comprises a handle assembly operably connected to the catheter tube, the handle assembly comprising an actuator configured to selectively apply and release tension to the at least one embedded control wire.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a schematic illustration of a medical device in proximity to electromagnets.

FIG. 2 is a schematic illustration of flexible catheter.

FIG. 3 is a shows a cross-section of a catheter with embedded components that enable the movement of the catheter.

FIG. 4 shows the tip/edge of a medical device/tool.

For simplicity and clarity of illustration, elements shown in the figures are not necessarily drawn to scale, and the dimensions of some elements may be exaggerated relative to other elements. In addition, reference numerals may be repeated among the figures to indicate corresponding or analogous elements.

### DETAILED DESCRIPTION

The invention provides a medical tool system coupled with vibration to improve penetration. The components of the system are: The inserted tool (either a tethered tool—e.g. a needle or a catheter) or an untethered tool. The tool diameter is between 0 mm and 1 cm and its length is between 100 micron (untethered) and 5 ft for a tethered tool, an external control system allowing vibrating the tip of the inserted tool in the direction orthogonal to the axis of insertion. The dynamic range of vibration is between 0.1 Hz and 1000 Hz and the dynamic range of tip motion is +−5 cm.

Optionally, the external control system may have a mechanism to advance the tool axially in coordination with the side vibrations.

In one embodiment the invention provides a dynamic medical tool for use in an individual, the dynamic medical tool comprising:
a medical tool comprising a distal end;
an element comprised in the medical tool;
wherein the element comprises a magnetic component, an actuator, or a combination thereof; and
wherein the element is configured to induce motion of the medical tool.

As used herein the term “dynamic” refers to any motion of the medical tool, in particular to one that is induced by various means of actuation, as described herein. The two primary means of actuation are: 1) a magnetic component comprised within a medical tool which is actuated at a distance with an external magnetic inducing apparatus or system, or, 2) an actuator comprised within the medical tool itself, which induces motion of the medical tool. The element being comprised in the medical tool can refer to placement anywhere in the medical tool. For example, on the surface of the medical tool, inside the body of the medical tool, in partial contact with the medical tool, etc.

As used herein the “medical tool” refers to any medical tool, device, implement, or their components, which interacts in any way into a patient or individual. For example, the insertion of the medical tool can be through tissue or through any channel or passage. Examples of penetration through tissue include, but are not limited to: skin, subcutaneous tissue, muscle tissue, membrane, ligaments, blood vessels, organs, bone, tumors, sacs, lymph nodes, cartilage. Examples of channels and passages include, but are not limited to: urethra, blood vessels, heart, valves, digestive tract, respiratory tract, bladder, bile duct, throat, nasal passages, rectum colon, lungs, abdomen, and joints. Insertion can also refer to motion through a non-biological structures. Examples of non-biological structures include, but are not limited to: IV bags, tubing, artificial membranes, implanted devices, synthetic stoppers, injection ports, prostheses, implants, bone cement, stents, artificial valves, internal monitoring devices, prosthetic limbs, and surgical mesh.

Thus a “dynamic medical tool” as defined herein, refers to a medical tool with means to move. Typically, the means are provided by actuation via various elements, as described herein.

The medical tool typically comprises a distal end, namely the end which is in contact with the individual. The terms “individual”, “patient” and “person” are used interchangeably when referring to the use of the dynamic medical tool for use in a particular application.

As used herein the term “element” refers to any structure comprised in the medical tool that enables motion in the medical tool. In one embodiment the element comprises a magnetic component, an actuator, or a combination thereof. The magnetic component and the actuator being means to achieve motion in the medical tool. In one embodiment the element further comprises a cutting means. In one embodiment the element further comprises a cutting tool. In one embodiment the element further comprises a penetrative element. In one embodiment the element further comprises an incising edge. The element being comprised in the medical tool can be described in any number of ways such as being attached to, affixed to, assembled on, disposed on, etc. These terms can be understood interchangeably, depending on the particular configuration of the dynamic medical tool. For example, if the element is attached to the surface of the dynamic medical tool, it can be correspondingly understood as being disposed on the surface or affixed thereon, etc.

In one embodiment the medical tool is selected from: catheter, magnetic capsule robot, endoscope, hypodermic needle, hollowed needle, solid needle, trocar, cannula, scalpel, catheter, implant, stent, ultrasound probe, temperature probe, medical tube, cauterizing probe, and medical drill. Magnetic capsule robots are used for capsule endoscopy and are typically propelled through the body by external magnetic fields allowing them to move through the tissue or channels.

In one embodiment the motion is selected from: vibration, linear motion, rotation, back-and-forth, circular, helical, pulsating, axial, lateral, and longitudinal. In one embodiment the motion is diametric. As used herein “diametric” refers to the movement of the tool's tip in a back-and-forth motion. For example, along two perpendicular axes that intersect at the center of the tool. The tool can vibrate along the wide axis or along the narrow axis (along the tool's length), depending on the operation.

In one embodiment the element is located on the distal end of the medical tool, or in close proximity to the distal end of the medical tool. In one embodiment the element is comprised on the tip of the medical tool. In one embodiment the distal end of the medical tool is the tip of the medical tool.

The element can be comprised on the surface of the medical tool, inside the medical tool, or partially embedded within the medical tool. For example, regarding a catheter, the element can be on the outer surface of the catheter, the inner surface of the catheter, inside the tubing of the catheter, or inside the catheter and partially extruding from either the outer surface or the inner surface of the tubing.

In one embodiment the range of motion of the distal end ranges between 0.1 mm to 5 cm. In one embodiment the range of motion of the distal end ranges between 0.1 mm to 2 cm. In one embodiment the range of motion of the distal end ranges between 0.1 mm to 1 cm. In one embodiment the range of motion of the distal end ranges between 0.1 mm to 5 mm. In one embodiment the range of motion of the distal end ranges between 0.5 mm to 5 mm.

In one embodiment the magnetic component is selected from: permanent magnet, non-permanent magnet, and electromagnet, or a combination thereof. In one embodiment the permanent magnet comprises any of the following selected from: neodymium, samarium-cobalt, alnico, iron, steel, cobalt, and ceramic magnet.

In one embodiment the dynamic medical tool further comprises a friction-minimizing coating. Examples of friction-minimizing coatings include, but are not limited to: silicone coating, hydrophilic coating, polytetrafluoroethylene (PTFE) coating, fluoropolymer coating, polyurethane coating, metallic coating, lubricant, and polymer.

In one embodiment the dynamic medical tool comprises any of the following selected from: polymer, metal, ceramic, composite, biodegradable material, glass, rubber, silicone, elastomer, textile, and hydrogels. In one embodiment the polymer comprises any of the following selected from: polyethylene (PE), polypropylene (PP), polyvinyl chloride (PVC), polymethyl methacrylate (PMMA), polycarbonate (PC), nylon, polyurethane (PU), silicone, polylactic acid (PLA), and polytetrafluoroethylene (PTFE).

In one embodiment the medical tool has a width ranging between 0.1 mm and 2 cm. The width refers to either the body of the medical tool, or the tip of the medical tool. In one embodiment the length of the medical tool ranges between 100 microns and 2 m. In some embodiments the medical tool is tethered. In one embodiment the medical tool is untethered.

The purpose of the induced motion of the medical tool is multi-faceted. Some of the benefits include: minimizing tissue trauma, enhanced precision, improve patient comfort, facilitate controlled depth, promote rapid healing, reduce friction, reduce scarring, enhanced sterility, prevention of biofilm formation, improve durability, reduce insertion/penetration force, facilitate fluid flow, enhanced tissue retraction, etc. In one embodiment the induced motion reduces friction, enhances ease of penetration, or a combination thereof, when the dynamic medical tool is in contact with the individual. As stated hereinabove, the medical tool can be used to penetrate tissue and/or enhance motion through a passage or channel. As such, “in contact with the individual” refers to any type of contact whether it results in tissue penetration or whether it results the passage of the medical tool through a passage/channel. As used herein the terms “insertion” and “penetration” are understood as the medical tool going from one medium to another or carrying out a medical procedure. Examples include: from air into a passage/channel, from air into tissue, from one type of tissue to another type of tissue, etc. For example, penetration can also include ‘tissue spreading’ or ‘tissue slicing’ whereby the tool is in contact with a tissue and the motion of the tool results in separation or movement of tissue.

In one embodiment the dynamic medical tool is disposable.

The dynamic medical tool is used in a system to achieve the induced motion and any of the stated results described herein.

In one embodiment the invention provides a dynamic insertion system comprising:
the dynamic medical tool of the invention;
a controller configured to communicate with the dynamic medical tool; and
a power source.

In one embodiment the power source and controller are configured to operate the actuator to achieve the induced motion. The power source can be selected from: mains, battery, or a combination thereof.

In one embodiment the system further comprises a magnetic inducing apparatus configured to be operated to generate a varying magnetic field, thereby remotely controlling the motion of the dynamic medical tool. The magnetic inducing apparatus comprises a plurality of coils. In one embodiment the magnetic field operates at a frequency ranging between 1 Hz to 5 kHz. In one embodiment the magnetic field operates at a frequency ranging between 100 Hz to 5 kHz. In one embodiment the magnetic field operates at a frequency ranging between 1 kHz to 5 kHz. In one embodiment the magnetic field operates at a frequency ranging between 1 kHz to 3 kHz.

In one embodiment the actuator operates at a frequency ranging between 1 Hz to 5 kHz. In one embodiment the actuator operates at a frequency ranging between 100 Hz to 5 kHz. In one embodiment the actuator operates at a frequency ranging between 1 kHz to 5 kHz. In one embodiment the actuator operates at a frequency ranging between 1 kHz to 3 kHz.

In one embodiment the power source is located externally to the dynamic medical tool, comprised within the dynamic medical tool, or a combination thereof. In one embodiment the system further comprises wireless communication configured between the controller and the dynamic medical tool. The controller is configured to provide any of the following, but not limited to: data collection, data transfer, signal processing, parameter adjustment, real-time monitoring, user interface management, safety monitoring and protection, automated adjustments, user feedback, power management, data analysis, diagnostics, and communication with external systems. Some of the parameters that the controller utilizes for applications used in the dynamic medical tool and related systems include, but are not limited to: frequency, amplitude, waveform type, duty cycle, duration of motion/vibrations, amplitude modulation, beat frequency, phase shift, power supply voltage control, temperature control, feedback from sensors, modulation of frequency or amplitude, user inputs, and control settings. In one embodiment the system further comprises at least one sensor. The at least one sensor can be comprised with the dynamic medical tool, or external to it. Examples of sensors include, but are not limited to: accelerometer, force sensor, temperature sensor, pressure sensor, capacitive sensor, electro-optic sensor, electromyographic (EMG) sensor, and tissue impedance sensor.

Medical tools can also include a magnetic capsule robot i.e., one that is already magnetized.

In one embodiment the invention provides a dynamic medical tool system for use in an individual, the dynamic medical tool comprising:
a magnetic capsule robot;
a magnetic inducing apparatus configured to be operated to generate a varying magnetic field, thereby remotely causing the magnetic capsule robot to vibrate;
a controller; and
a power source.

In one embodiment the vibration reduces friction, enhances ease of penetration, or a combination thereof, when the magnetic capsule robot is in contact with the individual. In one embodiment the magnetic field operates at a frequency ranging between 1 Hz to 5 kHz. In one embodiment the frequency is set at a value that reduces friction, enhances ease of penetration, or a combination thereof, This can be different depending on the circumstances of the contact between the magnetic capsule robot (or the dynamic medical tool generally) and the nature of the contact with the patient.

In one embodiment the magnetic capsule robot further comprises a wireless communication module configured to transfer data between the magnetic capsule robot and the controller. Wireless communication modules are configured to transmit and/or receive data between the catheter and an external control system, wherein the data includes information related to medical tool positioning, operational parameters, or feedback signals.

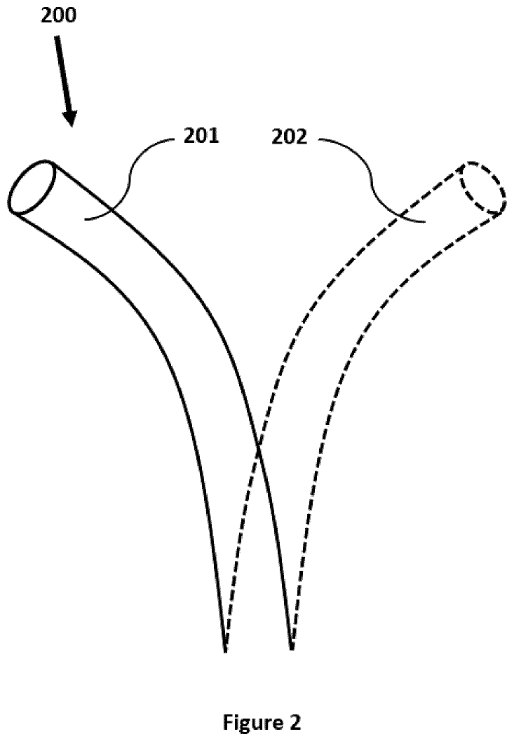

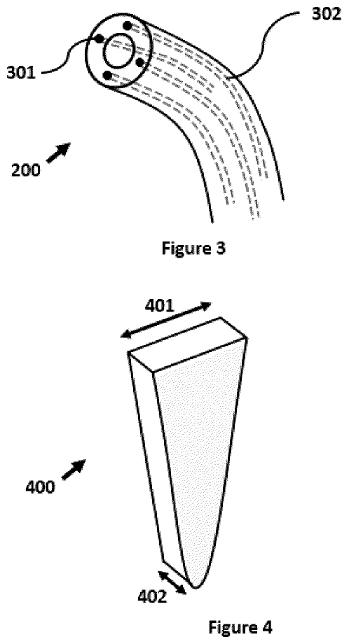

The invention provides a dynamic catheter that is configured to steer, at least, the tip of the catheter, whilst also enabling the diametric motion of the catheter tip. Means of vibration are provided herein to enable the vibration of the tip of the catheter for ease of penetration. FIG. 2 and FIG. 3 show a dynamic catheter, according to some embodiments.

In one embodiment the invention provides a dynamic catheter comprising:
a catheter tube;
at least one embedded control wire extending longitudinally along the central axis of the catheter tube wire and configured to deflect the distal tip of the catheter tube upon application of tension to the at least one embedded control wire;
a vibrating element coupled to the at least one embedded control wire, the catheter tube, or a combination thereof, and configured to induce vibrations at the distal end of catheter tube.

As understood to herein a “dynamic catheter” refers to a catheter that moves by providing external means. Such means can include, but is not limited to: control wires and a coupled vibration element.

As understood herein a “control wire” refers to a thin, flexible wire embedded within or routed through a catheter, configured to transmit mechanical force from an actuator, such as a handle assembly, to the distal tip of the catheter. By selectively applying tension to the control wire, the distal tip can be deflected, bent, or steered in one or more directions. Control wires are typically made of biocompatible materials, such as stainless steel or nitinol, to ensure durability, flexibility, and compatibility with medical procedures. The embedded wires can be sheath-guided or integrated into the material of the catheter itself.

In one embodiment the dynamic catheter further comprises a handle assembly operably connected to the catheter tube, the handle assembly comprising an actuator configured to selectively apply and release tension to the at least one embedded control wire.

### EXAMPLES

### Example 1

### Magnetic Implementation

The system consists of three magnetic coils. Two coils, A and B, are positioned on opposite sides of the tool along its diameter, while the third coil, C, is placed in front of the tool along its axis. When coil C is activated, it creates a magnetic field that pushes the tool forward into tissue, using the tool's magnetic properties. Meanwhile, activating coils A and B causes the tool to vibrate side-to-side along its diameter.

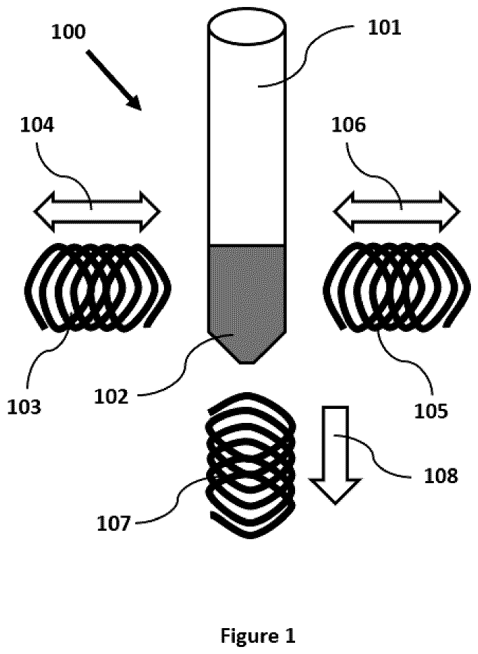

FIG. 1 shows a dynamic medical tool system 100 with a dynamic medical tool 101 and a plurality of coils 103, 105, and 107. Coil 103 induces lateral motion 104 for the dynamic medical tool 101; coil 105 induces lateral motion 106 for the dynamic medical tool 101, and coil 107 induces axial motion 108 for the dynamic medical tool. The dynamic medical tool 101 can comprise a tip 102 shown in grey with a magnetic element disposed thereon which enables the dynamic motion of the dynamic medical tool 101 when the plurality of coils are operated.

### Example 2

### Tethered Non-magnetic Implementation

The tool is a catheter with a flexible tip that can be controlled through cables embedded within the walls of the catheter. These cables are connected to a control unit that is integrated into the control handle. By pulling the cables in an alternating sequence, the control unit creates a diametric vibration at the tip of the catheter. This vibration enhances the tool's ability to move more easily through tissue, improving its performance during insertion or manipulation inside the body. The system allows for precise control over the catheter's motion, providing smoother and more effective navigation through complex tissue structures, ultimately benefiting the procedure and patient comfort. The design ensures flexibility and responsiveness, adapting to different medical scenarios with ease.

FIG. 2 shows a catheter 200 whereby the motion of the catheter can be manipulated by non-magnetic means. The catheter 200 can be in one position 201 and moved to another position 202 by these means.

FIG. 3 shows a cross-section of a flexible catheter 200 with four embedded cables 301 passing along the body of the tubing 302. Although FIG. 3 shows four embedded cables 301, any number of embedded cables can be used. Alternate activation of diametrically opposed pull-cables lead to diametric vibration of the flexible tip.

### Example 3

### Optimized Vibration Modes

FIG. 4 shows an example of the inserted tool tip 400 having a narrow axis 402 and a wide axis 401, both orthogonal to the main direction of insertion (analogous to a blade). In this case the diametric vibration allows for two distinct modes of motion:
1) Tissue slicing. vibration along the wide axis, while applying forward pressure, which means the pressure is applied along the narrow dimension ahead of the tool. This allows for maximization of the pressure at the cutting surface and slicing of the tissue in front of the tool;
2) Tissue spreading. vibration is provided along the narrow axis, without forward pressure, which means the wide surface on the tool is pressing on the tissue around it and spreading it aside, akin to blunt dissection surgical technique. This allows spreading the tissue layers as the tool is advanced, without actually slicing the tissue ahead of the tool.

Both types of diametric vibration can be combined with axial advancement of the tool as well as with axial vibration to lower the effective axial force needed to insert the tool into tissue, while maintaining a safe advancement profile and speed.

In one embodiment, the term “a” or “one” or “an” refers to at least one. In one embodiment the phrase “two or more” may be of any denomination, which will suit a particular purpose. In one embodiment, “about” or “approximately” may comprise a deviance from the indicated term of +1%, or in some embodiments, −1%, or in some embodiments, ±2.5%, or in some embodiments, ±5%, or in some embodiments, ±7.5%, or in some embodiments, ±10%, or in some embodiments, ±15%, or in some embodiments, ±20%, or in some embodiments, ±25%.

Those skilled in the art to which this invention pertains will readily appreciate that numerous changes, variations, and modifications can be made without departing from the scope of the presently disclosed subject matter, mutatis mutandis.

1. A dynamic medical tool for use in an individual, the dynamic medical tool comprising:
a medical tool comprising a distal end;
an element comprised in the medical tool;
wherein the element comprises a magnetic component, an actuator, or a combination thereof; and
wherein the element is configured to induce motion of the medical tool.

2. The dynamic medical tool of claim 1 wherein the medical tool is selected from: catheter, magnetic capsule robot, endoscope, hypodermic needle, hollowed needle, solid needle, trocar, cannula, scalpel, catheter, implant, stent, ultrasound probe, temperature probe, medical tube, cauterizing probe, and medical drill.

3. The dynamic medical tool of claim 1 wherein the motion is selected from: vibration, linear motion, rotation, back-and-forth, circular, helical, pulsating, axial, lateral, and longitudinal.

4. The dynamic medical tool of claim 1 wherein the element is located on the distal end of the medical tool, or in close proximity to the distal end of the medical tool.

5. The dynamic medical tool of claim 1 wherein the range of motion of the distal end ranges between 0.1 mm to 5 cm.

6. The dynamic medical tool of claim 1 wherein the magnetic component is selected from: permanent magnet, non-permanent magnet, and electromagnet, or a combination thereof.

7. The dynamic medical tool of claim 6 wherein the permanent magnet comprises any of the following selected from: neodymium, samarium-cobalt, alnico, iron, steel, cobalt, and ceramic magnet.

8. The dynamic medical tool of claim 1 further comprising a friction-minimizing coating.

9. The dynamic medical tool of claim 1 wherein the medical tool has a width ranging between 0.1 mm and 2 cm.

10. The dynamic medical tool of claim 1 wherein the length of the medical tool ranges between 100 microns and 2 m.

11. The dynamic medical tool of claim 1 wherein the induced motion reduces friction, enhances ease of penetration, or a combination thereof, when the dynamic medical tool is in contact with the individual.

12. The dynamic medical tool of claim 1 wherein the dynamic medical tool is disposable.

13. A dynamic medical tool system comprising:
the dynamic medical tool of claim 1;
a controller configured to communicate with the dynamic medical tool; and
a power source.

14. The system of claim 13 wherein the power source and controller are configured to operate the actuator to achieve the induced motion.

15. The system of claim 13 further comprising a magnetic inducing apparatus configured to be operated to generate a varying magnetic field, thereby remotely controlling the motion of the dynamic medical tool.

16. The system of claim 13 wherein the magnetic field operates at a frequency ranging between 1 Hz to 5 kHz.

17. The system of claim 13 wherein the actuator operates at a frequency ranging between 1 Hz to 5 kHz.

18. The system of claim 13 wherein the power source is located externally to the dynamic medical tool, comprised within the dynamic medical tool, or a combination thereof.

19. A dynamic medical tool system for use in an individual, the dynamic medical tool comprising:
a magnetic capsule robot;
a magnetic inducing apparatus configured to be operated to generate a varying magnetic field, thereby remotely causing the magnetic capsule robot to vibrate;
a controller; and
a power source.

20. The system of claim 19 wherein the vibration reduces friction, enhances ease of penetration, or a combination thereof, when the magnetic capsule robot is in contact with the individual.

21. The system of claim 19 wherein the magnetic field operates at a frequency ranging between 1 Hz to 5 kHz.

22. The system of claim 19 wherein the magnetic capsule robot further comprises a wireless communication module configured to transfer data between the magnetic capsule robot and the controller.

23. A dynamic catheter comprising:
a catheter tube;
at least one embedded control wire extending longitudinally along the central axis of the catheter tube wire and configured to deflect the distal tip of the catheter tube upon application of tension to the at least one embedded control wire;
a vibrating element coupled to the at least one embedded control wire, the catheter tube, or a combination thereof, and configured to induce vibrations at the distal end of catheter tube.

24. The dynamic catheter of claim 23 further comprising a handle assembly operably connected to the catheter tube, the handle assembly comprising an actuator configured to selectively apply and release tension to the at least one embedded control wire.
